(19) United States
(12) Patent Application Publication
(10) Pub. No.: US 2023/0242930 A1
(43) Pub. Date: Aug. 3, 2023

(54) METHOD FOR INCREASING METHIONINE CONTENT IN GRAINS BY GENE KNOCKOUT

(71) Applicant: Biotechnology Research Institute, CAAS, Beijing (CN)

(72) Inventors: Ling Jiang, Beijing (CN); Chunyi Zhang, Beijing (CN); Ying Ren, Beijing (CN); Tong Lian, Beijing (CN); Ji'an Liu, Beijing (CN)

(21) Appl. No.: 18/055,765

(22) Filed: Nov. 15, 2022

(30) Foreign Application Priority Data
Jan. 29, 2022 (CN) 202210110992.8

(51) Int. Cl. C12N 15/82; C12N 9/22; C12N 15/11

(52) U.S. Cl. CPC C12N 15/8253; C12N 9/22; C12N 15/11; C12N 2310/20

(57) ABSTRACT

The present disclosure provides a method for increasing a methionine content in grains by gene knockout. A maize ZmMETS2 gene is knocked out by a CRISPR/Cas9 technology, and it is found that the methionine content in maize grains is increased to a certain extent after the gene is knocked out. In the present disclosure, the CRISPR/Cas9 target site of the maize ZmMETS2 gene is designed and a function of the gene is found to significantly affect the methionine content. This provides a reference for CRISPR researches of the gene in maize and other crops, and provides a theoretical basis for methionine metabolism of the maize and other crops. Accordingly, the deficiency of human amino acid nutrition is alleviated by increasing the methionine content of transgenic plants.

### CROSS REFERENCE TO RELATED APPLICATION

This patent application claims the benefit and priority of Chinese Patent Application No. 202210110992.8, filed with the China National Intellectual Property Administration on Jan. 29, 2022, the disclosure of which is incorporated by reference herein in its entirety as part of the present application.

### REFERENCE TO SEQUENCE LISTING

The present application is being filed along with a Sequence Listing in electronic format. The Sequence Listing is provided as a file entitled SeqList-BGI010-001AUS, created Nov. 14, 2022, which is approximately 28,651 bytes in size. The information in the electronic format of the Sequence Listing is incorporated herein by reference in its entirety.

### TECHNICAL FIELD

The present disclosure belongs to the technical field of genetic engineering and molecular biology, and particularly relates to a method for increasing a methionine content in grains by gene knockout.

### BACKGROUND

Methionine, as an essential amino acid that cannot be synthesized by humans and monogastric animals, must be taken from the diet. On the one hand, methionine is a basic constituent amino acid of proteins, and its codon serves as the start codon to initiate translation of mRNAs for proteins. On the other hand, methionine indirectly regulates various cellular processes, including DNA and protein methylation, cell proliferation and differentiation, apoptosis, homeostasis, and gene expression, through S-adenosylmethionine as its major catabolite. Methionine is also extremely important especially in development and germination of plant seeds. There are at least two ways of methionine synthesis in animals and plants. One is to form methionine and tetrahydrofolate under the action of a methionine synthase with 5-methyltetrahydrofolate in a multi-tailed form and homocysteine as substrates. The other way works as follows: in animals, methionine is formed under the action of a betaine homocysteine S-methyltransferase with betaine derived from choline as a substrate; and in plants, methionine is formed under the action of a homocysteine methyltransferase with S-methylmethionine and homocysteine as substrates. However, methionine is found to have a relatively low content in the crop seeds, especially legume seeds, limiting nutritional quality of the legumes; plant-based diets contain only 50% to 75% of the methionine required for a balanced diet. In predominantly vegetarian cultures, or in developing countries where plant-derived foods are predominant, low methionine levels in crops may lead to nonspecific reactions of protein deficiency in human beings, such as reduced resistance to diseases, decreased blood protein level, and intellectual and physical developmental delays in young children. Moreover, since methionine is one of the four main dietary sources of methyl in the human body, methionine deficiency may also lead to methylation-related diseases (such as fatty liver, atherosclerosis, neurological diseases, and tumors). Therefore, increasing the methionine content of crop seeds can alleviate the deficiency of human amino acid nutrition.

Genome editing technology is one of the powerful means to study plant gene function and improve crop varieties. CRISPR/Cas9 is a tool for specific DNA modification of target genes, and has become the most popular gene editing technology nowadays due to high efficiency, convenience, and wide range of application. The CRISPR/Cas9 system is mainly composed of a small guide RNA (sgRNA) and a Cas9 protein domain. After recognizing and cutting a target DNA double-strand, DNA mutations are generated through homologous recombination or non-homologous end joining repair. This technology can study, identify and screen genes related to important traits, and can design and modify crop varieties more accurately and quickly; in addition, this technology can aggregate multiple excellent traits in a targeted manner to improve resistance, yield, or quality of the plants. It has become one of the main methods of biofortification by improving an amino acid content of the plants through gene editing.

### SUMMARY

An objective of the present disclosure is to provide a method for increasing a methionine content in grains by gene knockout.

To achieve the above objective, in the present disclosure, knockout is conducted on a gene ZmMETS2 (the gene has a site number of GRMZM2G112149, with reference to a maize genome B72 RefGen_v2) encoding a methionine synthase 2 in maize by a gene editing technology; where a background material is derived from an inbred line B104, a primer pair for amplifying a CDS sequence includes a Primer1 (SEQ ID NO: 4)/a Primer2 (SEQ ID NO: 5), the CDS sequence is shown in SEQ ID NO: 1, an encoded amino acid has a sequence shown in SEQ ID NO: 2, and a specific preferred knockout target site is shown in SEQ ID NO: 3.

The method for increasing a methionine content in maize grains by gene knockout includes the following steps:

(1) mutation of target: selecting a target site in a genomic region corresponding to a ZmMETS2 exon, where a 3′-end has the characteristics of NGG (N is any base selected from the group consisting of A, T, C, and G);

(2) constructing a CRISPR/Cas9 vector containing the target site, namely a recombinant plasmid ZmMETS2-sgRNA-POsCas9 (where a construction method refers to Li C X, Liu C L et al., 2017, Plant Biotechnology Journal, 15: 1566-1576);

(3) introducing the CRISP/Cas9 vector into plant cells by conventional biotechnological methods, such as using a Ti plasmid, using a plant virus vector, direct DNA transformation, microinjection, and electroporation, to obtain a recombinant strain ZmMETS2-sgRNA-POsCas9 (Weissbach, 1998, Method for Plant Molecular Biology VIII, Academy Press, New York, pp. 411-463; Geiserson and Corey, 1998, Plant Molecular Biology, 2nd Edition);

(4) transforming the recombinant strain ZmMETS2-sgRNA-POsCas9 into a maize inbred line (variety B104) by an Agrobacterium-mediated method, to obtain gene-knockout transgenic TO generation plants;

(5) conducting self-pollination on the TO generation plants, and screening transgenic negative plants (ZmMETS2-KO) with stable inheritance that have gene mutation but do not contain the recombinant vector ZmMETS2-sgRNA-POsCas9 from obtained T1 generation plants; and

(6) according to the determination of amino acids in food (GB 5009.124-2016 National Food Safety Standard), detecting a methionine content in dry grains of gene-knockout transgenic maize using an amino acid analyzer, where a detection organ is harvested dried maize grains.

Primers for conducting the CRISPR/Cas9 knockout vector include a Primer3 (SEQ ID NO: 6) and a Primer4 (SEQ ID NO: 7). Primers for amplifying the CDS sequence of the ZmMETS2 gene in the inbred line B104 include a Primer1 (SEQ ID NO: 4) and a Primer2 (SEQ ID NO: 5); Primers for detecting whether the target site of the transgenic plant is mutated include a Primer7 (SEQ ID NO: 10) and a Primer8 (SEQ ID NO: 11); and qRT-PCR primers for detecting a plant transcript level include a Primer9 (SEQ ID NO: 12) and a Primer10 (SEQ ID NO: 13).

The present invention has the following beneficial effects: in the present disclosure, the gene ZmMETS2 encoding the methionine synthase 2 in maize is edited by the CRISPR/Cas9 technology, and it is found that the knockout of this gene increases the methionine content in maize grains to a certain extent. The methionine can be generated by the methionine synthase 2 encoded by the maize ZmMETS2 gene with homocysteine as a substrate. The loss of function in the enzyme results in a marked increase in a transcription level of key genes in other methionine synthesis, which ultimately leads to accumulation of the methionine. Therefore, the present disclosure provides a theoretical basis for studying amino acid metabolism in maize and other crops, and also improves the methionine content of transgenic plants by means of genetic engineering, thereby alleviating the deficiency of human beings in amino acid nutrition.

### BRIEF DESCRIPTION OF THE DRAWINGS

FIG. 1A is a PCR amplification result of a ZmMETS2 gene CDS sequence of a wild-type maize inbred line B104 in Example 1 of the present disclosure; FIG. 1B is a position (T1) of a CRISPR/Cas9 knockout target site designed on a ZmMETS2 gene exon by the present disclosure;

FIG. 2 shows a part of PCR amplification results of a Bar gene of a ZmMETS2 TO generation transgenic plant material in Example 1 of the present disclosure; where 1, 2, 3, 4, and 5 are positive transgenic plants, and WT is wild-type B104;

FIG. 3 shows results of a sequencing peak map of a wild-type B104 with a DNA sequence fragment set forth in SEQ ID NO: 22(A) and a gene-edited plant ZmMETS2-KO with a DNA sequence fragment set forth in SEQ ID NO: 23 (B) at a first target site in Example 1 of the present disclosure;

FIG. 4 shows a relative expression level of genes involved in methionine synthesis in transgenic maize grains detected by qRT-PCR in Example 2 of the present disclosure; where white columns represent a transcription level of a corresponding gene in leaves of the wild-type maize (B104), and the expression level in B104 is defined as 1; and black columns represent a transcript level of a corresponding gene in leaves of the transgenic lines (CR);

FIG. 5 shows methionine synthase enzymatic reactions of a maize ZmMETS2 protein using 5-methyltetrahydrofolate in three-tailed or four-tailed forms, respectively, as substrates in Example 3 of the present disclosure, with a detection means of a liquid chromatography-mass spectrometry system; where yeast methionine synthase METE has an enzymatic reaction rate shown as white columns, and ZmMETS2 has an enzymatic reaction rate shown as black columns; 5-M-Glu3 represents the three-tailed form of 5-methyltetrahydrofolate; and 5-M-Glu4 represents the four-tailed form of 5-methyltetrahydrofolate;

FIG. 6 shows detection results of the methionine content in transgenic maize dry grains in Example 4 of the present disclosure, where a white column represents the methionine level in wild-type maize dry grains (B104), and the other three columns (CR1/CR2/CR3) represent methionine levels in dry grain materials of different transgenic lines.

### DETAILED DESCRIPTION OF THE EMBODIMENTS

To facilitate the understanding of the present disclosure, the present disclosure will be described more comprehensively below. However, the present disclosure can be implemented in many different forms and is not limited to the examples described herein. On the contrary, these embodiments are provided to make the present disclosure more thoroughly and comprehensively understood.

Unless otherwise specified, the examples each are in accordance with conventional experimental conditions, such as a molecular cloning laboratory manual of Sambrook et al. (Sambrook J & Russell D W, Molecular Cloning: a Laboratory Manual, 2001), or in accordance with conditions suggested by the manufacturer's instructions.

### Example 1 Construction of a Maize ZmMETS2 Gene-Knockout Vector, and Acquisition and Identification of Transgenic Plants

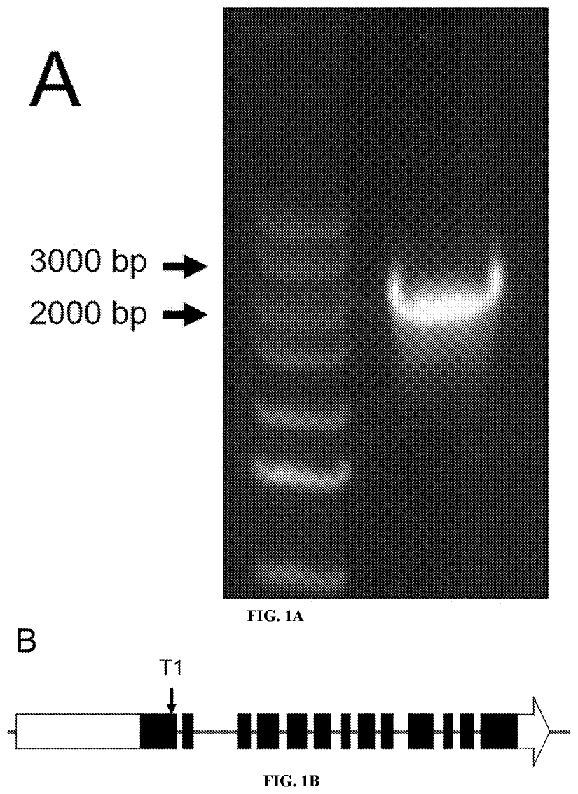

(1) The leaves of a maize inbred line B104 were collected, a total RNA of the maize was extracted by a Trizol method, and a maize cDNA was obtained by a RevertAid FirstStrand cDNA Synthesis Kit (Thermo). The full-length CDS of a GRMZM2G112149 gene was amplified using the maize cDNA as a template and Primer1 (SEQ ID NO: 4)/Primer2 (SEQ ID NO: 5) as amplification primers (FIG. 1A).

(2) Referring to sequencing results, a target site was designed in a genomic region corresponding to a ZmMETS2 exon (T1 in FIG. 1B, a design website was: http://cas9.cbi.pku.edu.cn/index.jsp), a 3′-end had NGG (N was any base selected from the group consisting of A, T, C, and G), with a sequence shown in SEQ ID NO: 3.

(3) Referring to a construction method of a gene knockout vector (Li C X, Liu C L et al., 2017, Plant Biotechnology Journal, 15: 1566-1576), a CRISPR/Cas9 vector containing a target site was constructed using primers Primer3 (SEQ ID NO: 6) and Primer4 (SEQ ID NO: 7) included target sequences, namely a recombinant plasmid ZmMETS2-sgRNA-POsCas9.

(4) The recombinant plasmid ZmMETS2-sgRNA-POsCas9 was sent to Beijing Bomei Xingao Technology Co., Ltd. for transformation, to obtain ZmMETS2 gene-edited transgenic T0 generation plants.

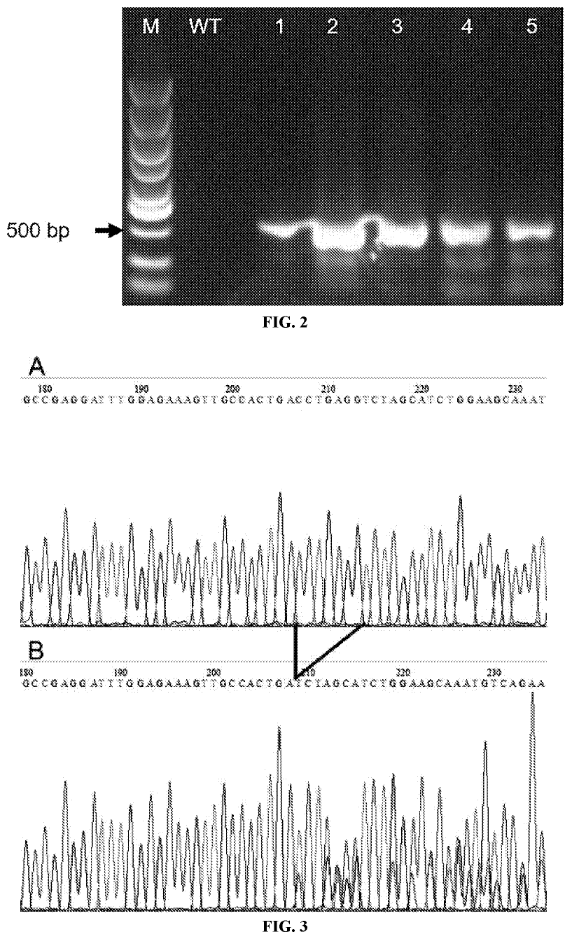

(5) Whether the TO generation plants had transgenes was identified using a marker gene BAR on the recombinant plasmid ZmMETS2-sgRNA-POsCas9 vector, and an upstream primer Primer5 (SEQ ID NO: 8) and a downstream primer Primer6 (SEQ ID NO: 9) were designed for BAR gene detection. The PCR reaction system included: 1 μl of a DNA template, 0.5 μl of the Primer5 (SEQ ID NO: 8), 0.5 μl of the Primer6 (SEQ ID NO: 9), 10 μl of Taq-Mix (purchased from Beijing GenStar Biotechnology Co., Ltd.), and 8 μl of ddH₂O; a PCR amplification program included: initial denaturation at 95° C. for 1 min; denaturation at 95° C. for 30 sec, annealing at 55° C. for 30 sec, and extension at 72° C. for 60 sec, conducting 35 cycles; and final extension at 72° C. for 10 min, and incubation at 4° C. The detection results were shown in FIG. 2, where 1, 2, 3, 4, and 5 were positive transgenic plants.

(6) Primers Primer7 (SEQ ID NO: 10) and Primer8 (SEQ ID NO: 11) were designed on a ZmMETS2 genome sequence to detect whether T1 generation plants were mutated at the target site. Amplification was conducted using a DNA of the T1 generation plants as a template. A PCR reaction system included: 1 μl of a DNA template, 0.5 μl of the Primer7 (SEQ ID NO: 10), 0.5 μl of the Primer8 (SEQ ID NO: 11), 0.5 μl of Fast-Pfu (purchased from Beijing TransGen Biotech Co., Ltd.), 2 μl of dNTP, 5 μl of 5×Buffer, and 16 μl of ddH₂O; a PCR amplification program included: initial denaturation at 95° C. for 1 min; denaturation at 95° C. for 20 sec, annealing at 55° C. for 20 sec, and extension at 72° C. for 2 min, conducting 40 cycles; and final extension at 72° C. for 10 min, and incubation at 4° C. The sequencing results were shown in FIG. 3, where FIG. 3A was a sequence of the target site of wild-type B104; in FIG. 3B, a base deletion (CCTGAGG) occurred at the target site, resulting in premature termination of amino acid translation, and the ZmMETS2 gene was knocked out.

ZmMETS2 gene knockout: homozygous mutation-deletion

AGAAAGTTGCCACTGA CCTGAGG TCTAGC (WT, SEQ ID
NO: 20)
AGAAAGTTGCCACTGA ------- TCTAGC (ZmMETS2-KO,
SEQ ID NO: 21).

### Example 2 Verification of Activation of Genes Involved in Methionine Synthesis in Gene-Edited Plants at the Transcriptional Level

There are at least two ways of methionine synthesis in plants. One way is to form methionine and tetrahydrofolate under the action of a methionine synthase with 5-methyltetrahydrofolate in a multi-tailed form and homocysteine as substrates, and the methionine synthase gene ZmMETS2 also has homologous genes ZmMETS1 and ZmMETS3. Another way includes: methionine is formed under the action of a homocysteine methyltransferase with S-methylmethionine and homocysteine as substrates, with an encoding gene ZmHMT. Therefore, after ZmMETS2 gene was edited, transcription levels of key genes involved in methionine synthesis of transgenic plants was detected using a qRT-PCR detection kit (purchased from Beijing TransGen Biotech Co., Ltd.).

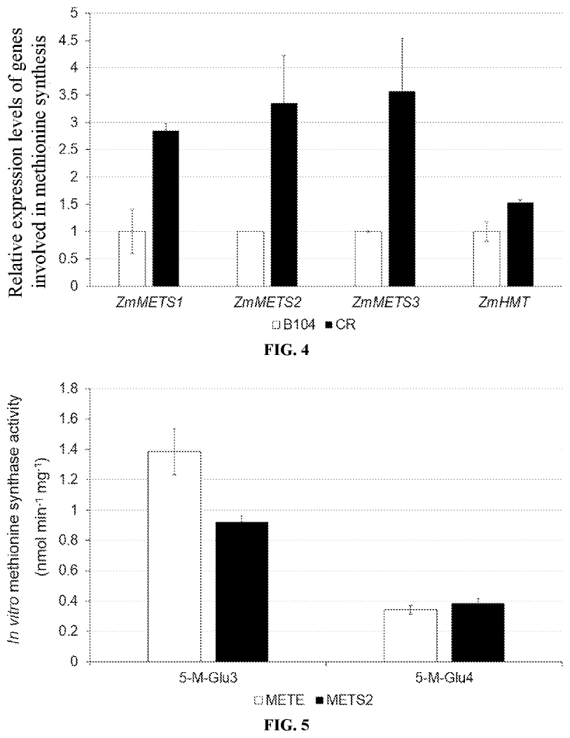

After the TO generation grains were planted, negative transgenic plants with ZmMETS2 gene knockout and without the CRISPR/Cas9 recombinant plasmid were isolated, namely stably-inherited ZmMETS2 gene-knockout T1 generation lines. The ZmMETS2 gene-knockout T1 generation plants were self-pollinated to obtain T2 generation seeds. After 30 d of seed germination, leaves were frozen in liquid nitrogen and ground into powder; a total RNA was extracted with a total RNA extraction kit (purchased from Beijing Yuanpinghao Biotechnology Co., Ltd.), and a cDNA was obtained with a RevertAid First Strand cDNA Synthesis Kit (Thermo). The cDNA was detected by a qRT-PCR detection kit (purchased from Beijing TransGen Biotech Co., Ltd.). A PCR reaction system of the ZmMETS2 gene included: 1 μl of a DNA template, 0.4 μl of the Primer9 (SEQ ID NO: 12), 0.4 μl of the Primer10 (SEQ ID NO: 13), 0.4 μl of a Dye II, 10 μl of a 2× Buffer, and 7.8 μl of ddH₂O; a PCR amplification program included: initial denaturation at 94° C. for 30 sec; denaturation at 94° C. for 5 sec, and annealing at 60° C. for 30 sec, conducting 40 cycles; and incubation at 4° C. The same procedure was used for ZmMETS1, ZmMETS3, and HMT with primers Primer11 (SEQ ID NO: 14) and Primer12 (SEQ ID NO: 15), Primer13 (SEQ ID NO: 16) and Primer14 (SEQ ID NO: 17), and Primer15 (SEQ ID NO: 18) and Primer16 (SEQ ID NO: 19), respectively. The detection results were shown in FIG. 4. Compared with wild-type B104, the transcription levels of ZmMETS1, ZmMETS2, ZmMETS3, and ZmHMT genes were significantly increased in transgenic plants.

### Example 3 Verification of ZmMETS2 Eenzymatic Reaction Experiments with Three-Tailed or Four-Tailed 5-Methyltetrahydrofolate as Substrates, Respectively

(1) A yeast methionine synthase METE gene and a maize ZmMETS2 gene CDS sequence were separately ligated into a PET28a vector, and transformed into BL21 E. coli. Induction was conducted overnight at 16° C. using 0.3 mM isopropylthiogalactoside.

(2) The cells were collected, homogenized, and a supernatant was purified with a Ni-NTA affinity column (Qiagen). After elution, an obtained protein was dialyzed with a dialysis bag, and concentrated by an ultrafiltration tube; and the protein was replaced in a protein buffer solution (including 150 mM NaCl and 25 mM Tris-HCl, pH 7.2), such that a protein concentration was about 1 mg mL⁻¹.

(3) With multi-tailed 5-methyltetrahydrofolate and homocysteine as substrates, a reaction was conducted at 30° C. for 30 min by the methionine synthase, and products were methionine and tetrahydrofolate. In this system, changes of a 5-methyltetrahydrofolate content reflected an activity of the methionine synthase. A 300 μl reaction system included 50 mM of potassium phosphate, 50 mM of Tris-HCl at pH 8.0, 0.05 mM of homocysteine, 0.05 mM of 5-methyltetrahydrofolate in a three-tailed or four-tailed form, and 10 μl of a purified enzyme. After the reaction, 700 μl of a reaction stop solution (including 50% methanol, 0.1% sodium ascorbate, 0.5% β-mercaptoethanol, and 20 mM ammonium acetate) was added.

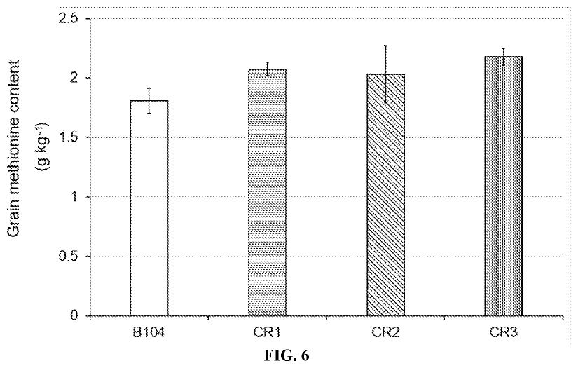

(4) The 5-methyltetrahydrofolate in three-tailed or four-tailed form was detected using liquid chromatography-mass spectrometry, contents were compared before and after the reaction, and an activity of the methionine synthase was calculated. When the three-tailed 5-methyltetrahydrofolate was used as a substrate, the activity of maize ZmMETS2 was 66% that of yeast METE; in contrast, when the four-tailed 5-methyltetrahydrofolate was used as a substrate, the activity of maize ZmMETS2 was 112% that of yeast METE (FIG. 5). Therefore, ZmMETS2 has a methionine synthase activity varied with the form of the substrate tails similar to the yeast methionine synthase METE.

### Example 4 Determination of the Methionine Content in ZmMETS2 Gene-Knockout Maize

According to the determination of amino acids in food (GB 5009.124-2016 National Food Safety Standard), the methionine content was detected in dry grains of gene-knockout transgenic maize using an amino acid analyzer, where a detection organ was harvested dried maize grains.

The results were shown in FIG. 6. The methionine content in ZmMETS2 transgenic maize dry grains was increased by about 15% to 20% compared with the control (wild-type).

The above described are merely several embodiments of the present invention. Although these embodiments are described specifically and in detail, they should not be construed as a limitation to the patent scope of the present disclosure. It should be noted that those of ordinary skill in the art may further make several variations and improvements without departing from the idea of the present disclosure, but such variations and improvements should all fall within the protection scope of the present disclosure. Therefore, the protection scope of the present disclosure shall be subject to the protection scope defined by the claims.

1. A method for increasing a methionine content in grains by gene knockout, comprising: conducting knockout on a maize ZmMETS2 gene to obtain a maize plant with an increased methionine content in the grains.

2-6. (canceled)

7. The method for increasing a methionine content in grains by gene knockout according to claim 1, wherein the gene knockout comprises the following steps: constructing a recombinant gene CRISPR/Cas9 knockout vector containing a target sequence of the maize ZmMETS2 gene, transforming a wild-type maize inbred line, and conducting a mutation on the maize ZmMETS2 gene; and the target sequence has a nucleotide sequence shown in SEQ ID NO: 3.

8. The method for increasing a methionine content in grains by gene knockout according to claim 7, comprising constructing a CRISPR/Cas9 vector using primers, wherein the primers of the CRISPR/Cas9 knockout vector comprise a Primer3 and a Primer4, with nucleotide sequences shown in SEQ ID NO: 6 and SEQ ID NO: 7, respectively.


SEQUENCE LISTING (23 sequences)

SEQ ID NO: 1 - DNA, 2298 bp, synthetic construct
  source 1..2298: mol_type = other DNA; note = CDS sequence
atggcgtcccatattgttggataccctcgcatgggccccaagagggagctcaagtttgccttggagtctttctgggatgggaagagcagcgccgaggatttggagaaagttgccactgacctgaggtctagcatctggaagcaaatgtcagaagctgggatcaagtacattcccagcaataccttctcgtactacgaccaggttcttgataccacggccatgcttggcgctgtcccagagcgctactcttggactggaggcgagattggcttgagcacctacttctctatggccaggggaaatgccactgtccctgccatggagatgaccaagtggtttgatacaaactaccactttattgtccctgaacttggtccaagcaccaagttcacatacgcttctcacaaggctgtttctgagtacaaggaggcaaaggcgctcggcattgatacagtcccagtgcttgttggaccagtctcatacttgctcctctctaagcctgccaagggtgtggagaaatctttctctcttctttcacttcttggtagcattcttcccatctacaaggaggttgttgctgagctgaaggcagctggtgcttcatggattcagcttgatgagcctacccttgttaaagaccttgatgctcacgaattggccgcattctcttcagcatatgctgaactggagtcatcgttctctggattgaatgtgcttatcgagacatacttcgctgatattcctgctgagtcctacaagaccctcacatcattgagtggtgtgactgcttacggtttcgatcttatccgtggagccaagacccttgatcttatcaggagcagcttcccctctgggaagtacctcttcgctggtgttgtagatggacgcaacatttgggctgatgatcttgctgcatctcttagcactcttcattctcttgaggctgttgctggcaaggacaaacttgtggtgtcaacctcctgctcactgatgcacaccgctgttgaccttgtaaatgagactaagctggatgatgagattaagtcatggcttgcatttgctgcccaaaaggttgttgaggttaatgcccttgccaaggctttggcaggccaaaaggatgaggtctactttgcagccaatgctgctgctcaggcctcaaggagatcatcgcccagggtgacaaacgaggaggtccagaaggctgcagctgctttgaggggatctgaccaccgccgttctaccactgtttctgctagattggatgctcagcagaaaaagctcaaccttcctgtccttcccacaaccacaattggttcattccctcagactgtggaactcaggagggttcgccgtgaatacaaggcaaagaagatcaccgaggacgaatacatcagtgccatcaaggaagaaatcagcaaggtcgtcaagatccaagaggagcttgacattgatgtgcttgtgcatggagagccagagagaaatgacatggttgagtacttcggtgagcaattatctggttttgcgttcactgccaacggatgggtgcaatcctatggatcacgctgtgtgaagccacccattatctacggtgatgtcagccggccgaaccccatgactgttttctggtccaagatggcacagagcatgacccctcgtcccatgaagggaatgttgactggtccggtcacaatcctcaactggtcattcgtcaggaacgaccagcctaggtttgagacatgctaccaaatagctcttgcaatcaaaaaggaggttgaggatcttgaggctgctggtattcaggtgatccagatcgatgaggcagctctaagggagggtctgccactacgcaagtcagagcatgcattctacctggactgggctgtccactctttcaggatcaccaactgcggagtccaggacaccacccagatccacacccacatgtgctactccaacttcaacgacatcatccactccatcatcgacatggatgccgatgtgatcacgatcgagaactcccggtctgacgagaagctactgtccgtcttccgtgagggtgtgaagtacggagctggcattggccctggtgtctacgacatccactctcctaggattccctccacagaggagatcgcagaccgcgtcgagaagatgctcgccgtgctcgacaccaacatcctctgggtgaaccctgactgtggtctcaagacacgcaagtacacggaggtcaagcccgccctgaccaacatggtctcggccaccaagctcatccgcacccagcttgccagcgcgaaatga

SEQ ID NO: 2 - protein, 765 aa, synthetic construct
  source 1..765: note = Amino acid sequence encoded by the CDS sequence
MASHIVGYPRMGPKRELKFALESFWDGKSSAEDLEKVATDLRSSIWKQMSEAGIKYIPSNTFSYYDQVLDTTAMLGAVPERYSWTGGEIGLSTYFSMARGNATVPAMEMTKWFDTNYHFIVPELGPSTKFTYASHKAVSEYKEAKALGIDTVPVLVGPVSYLLLSKPAKGVEKSFSLLSLLGSILPIYKEVVAELKAAGASWIQLDEPTLVKDLDAHELAAFSSAYAELESSFSGLNVLIETYFADIPAESYKTLTSLSGVTAYGFDLIRGAKTLDLIRSSFPSGKYLFAGVVDGRNIWADDLAASLSTLHSLEAVAGKDKLVVSTSCSLMHTAVDLVNETKLDDEIKSWLAFAAQKVVEVNALAKALAGQKDEVYFAANAAAQASRRSSPRVTNEEVQKAAAALRGSDHRRSTTVSARLDAQQKKLNLPVLPTTTIGSFPQTVELRRVRREYKAKKITEDEYISAIKEEISKVVKIQEELDIDVLVHGEPERNDMVEYFGEQLSGFAFTANGWVQSYGSRCVKPPIIYGDVSRPNPMTVFWSKMAQSMTPRPMKGMLTGPVTILNWSFVRNDQPRFETCYQIALAIKKEVEDLEAAGIQVIQIDEAALREGLPLRKSEHAFYLDWAVHSFRITNCGVQDTTQIHTHMCYSNFNDIIHSIIDMDADVITIENSRSDEKLLSVFREGVKYGAGIGPGVYDIHSPRIPSTEEIADRVEKMLAVLDTNILWVNPDCGLKTRKYTEVKPALTNMVSATKLIRTQLASAK

SEQ ID NO: 3 - DNA, 23 bp, synthetic construct
  source 1..23: mol_type = other DNA; note = A specific preferred knockout target site
agaaagttgcctctgacctcagg

SEQ ID NO: 4 - DNA, 27 bp, synthetic construct
  source 1..27: mol_type = other DNA; note = Primer 1
atggattacaaggatgacgacgataag

SEQ ID NO: 5 - DNA, 25 bp, synthetic construct
  source 1..25: mol_type = other DNA; note = Primer 2
catttcgcgctggcaagctgggtgc

SEQ ID NO: 6 - DNA, 34 bp, synthetic construct
  source 1..34: mol_type = other DNA; note = Primer 3
cagtggtctcagccagaaagttgccactgacctg

SEQ ID NO: 7 - DNA, 34 bp, synthetic construct
  source 1..34: mol_type = other DNA; note = Primer 4
cagtggtctcaaaacaggtcagtggcaactttct

SEQ ID NO: 8 - DNA, 26 bp, synthetic construct
  source 1..26: mol_type = other DNA; note = Primer 5
gtctgcaccatcgtcaaccactacat

SEQ ID NO: 9 - DNA, 24 bp, synthetic construct
  source 1..24: mol_type = other DNA; note = Primer 6
tgaagtccagctgccagaaaccca

SEQ ID NO: 10 - DNA, 24 bp, synthetic construct
  source 1..24: mol_type = other DNA; note = Primer 7
ccaagatggcacagagcatgaccc

SEQ ID NO: 11 - DNA, 24 bp, synthetic construct
  source 1..24: mol_type = other DNA; note = Primer 8
cggacagtagcttctcgtcagacc

SEQ ID NO: 12 - DNA, 30 bp, synthetic construct
  source 1..30: mol_type = other DNA; note = Primer 9
taagattttactgttatatagaaagatggc

SEQ ID NO: 13 - DNA, 26 bp, synthetic construct
  source 1..26: mol_type = other DNA; note = Primer 10
gggacaataaagtggtagtttgtatc

SEQ ID NO: 14 - DNA, 22 bp, synthetic construct
  source 1..22: mol_type = other DNA; note = Primer 11
gatccaggacaccacccagatc

SEQ ID NO: 15 - DNA, 21 bp, synthetic construct
  source 1..21: mol_type = other DNA; note = Primer 12
ctcctcggaaacgaaaaaggc

SEQ ID NO: 16 - DNA, 24 bp, synthetic construct
  source 1..24: mol_type = other DNA; note = Primer 13
ctctacaaatgaaacccctcagac

SEQ ID NO: 17 - DNA, 22 bp, synthetic construct
  source 1..22: mol_type = other DNA; note = Primer 14
gtatccaacaatatgggacgcc

SEQ ID NO: 18 - DNA, 21 bp, synthetic construct
  source 1..21: mol_type = other DNA; note = Primer 15
ggaggcacaggcattggttga

SEQ ID NO: 19 - DNA, 23 bp, synthetic construct
  source 1..23: mol_type = other DNA; note = Primer 16
gagcacaggttcttaccgtccac

SEQ ID NO: 20 - DNA, 29 bp, synthetic construct
  source 1..29: mol_type = other DNA; note = DNA sequence of WO
agaaagttgccactgacctgaggtctagc

SEQ ID NO: 21 - DNA, 22 bp, synthetic construct
  source 1..22: mol_type = other DNA; note = DNA sequence of ZmMETS2-KO
agaaagttgccactgatctagc

SEQ ID NO: 22 - DNA, 56 bp, synthetic construct
  source 1..56: mol_type = other DNA; note = DNA sequence fragment of a wild-type B104
gccgaggatttggagaaagttgccactgacctgaggtctagcatctggaagcaaat

SEQ ID NO: 23 - DNA, 56 bp, synthetic construct; identical to SEQ ID NO: 22
  source 1..56: mol_type = other DNA; note = DNA sequence fragment of gene-edited plant ZmMETS2-KO